(19) United States
(12) Patent Application Publication
(10) Pub. No.: US 2003/0078209 A1
(43) Pub. Date: Apr. 24, 2003

(54) Solid compositions exhibiting iron binding activity

(76) Inventor: Richard J. Schmidt, Barnoldswick (GB)

Correspondence Address: Philip Johnson, Johnson & Johnson, One, Johnson & Johnson Plaza, New Brunswick, NJ, 08933-7003, US

(21) Appl. No.: 10/262,983

(22) Filed: Dec. 19, 2002

Related U.S. Application Data
(63) Continuation-in-part of application No. 09/569,555, filed on May 12, 2000, now abandoned.

(51) Int. Cl. A61K 38/39; A61K 31/717

(52) U.S. Cl. 514/12; 514/57

(57) ABSTRACT

The invention provides solid porous compositions, substantially insoluble in water, comprising at least 25% by weight of an oxidised cellulose and having a significant capacity to bind dissolved iron. The invention also provides a method of sequestering dissolved iron from aqueous environments by bringing said compositions into contact with said environments. The iron-binding property of oxidised cellulose may be used for the prevention or treatment of infections by iron-requiring micro-organisms such as bacteria and yeasts. It may also be used to treat chronic inflammatory lesions or processes where inappropriately available iron is acting as a catalyst for the generation of damaging reactive oxygen species.

This application is a continuation-in-part of U.S. application Ser. No. 09/569,555 Filed on May 12, 2000.

### FIELD OF THE INVENTION

The present invention relates to compositions containing oxidised cellulose that show a significant capacity to bind dissolved iron, processes suitable for the preparation of such compositions, and the use of such compositions in therapeutic applications.

### BACKGROUND OF THE INVENTION

In the complex biochemistry of infection, it is recognised that availability of iron is essential for the survival, replication, and differentiation of invading micro-organisms. Many micro-organisms can either secrete their own siderophores or utilise the siderophores secreted by other micro-organisms for the purpose of scavenging iron from their surroundings. This subject was discussed more fully by Weinberg E D in the Quarterly Review of Biology 64(3): 261-290 (1989) and later by Jurado R L in Clinical Infectious Diseases: An Official Publication of the Infectious Diseases Society of America 25(4): 888-895 (1997). It therefore appears that removal of iron (which may be present as decompartmentalised free Fe²⁺/Fe³⁺ ions or in weak association with a complexant) from damaged tissue could assist in the prevention and treatment of infection by micro-organisms such as bacteria and yeasts.

Van Asbeck B S and co-authors in the European Journal of Clinical Microbiology 2(5): 426-431 (1983) described how the iron chelator desferrioxamine could be used to inhibit bacterial multiplication. Desferrioxamine is a low molecular weight iron chelator and therefore its clinical or medical uses for the prevention or treatment of infection are limited to situations where the solubility of it or its clinically acceptable salts is a desirable characteristic.

Removal of decompartmentalised free Fe²⁺/Fe³⁺ ions or iron in weak association with a complexant is also beneficial in clinical or medical situations where persistent inflammation, increased connective tissue degradation, and lipid peroxidation are the result of said iron acting as a catalyst for the generation of damaging reactive oxygen species in cells and tissues. In the International Journal of Biochemistry and Cell Biology 27(2): 109-122 (1995), Morris C J and co-authors discussed the dangerous partnership of iron and reactive oxygen species. Further elaboration of this subject was provided by Wenk J and co-authors in the Journal of Investigative Dermatology 116(6): 833-839 (2001) where they described the preparation of a material comprising desferrioxamine covalently coupled to cellulose gauze for use as a dressing in the treatment of chronic venous leg ulcers. U.S. Pat. Nos. 5,217,998 (Hedlund B E et al., 1993) and 6,156,334 (Meyer-Ingold W et al., 2000) also describe the preparation and medical or clinical use of desferrioxamine covalently coupled to various soluble or insoluble support materials.

### SUMMARY OF THE INVENTION

The invention provides solid porous compositions, substantially insoluble in water, comprising at least 25% by weight of oxidised cellulose and having a significant capacity to bind dissolved iron. The invention also provides a method of sequestering dissolved iron from aqueous environments by bringing said compositions into contact with said environments. The iron-binding property of oxidised cellulose may be used for the prevention or treatment of infections by iron-requiring microorganisms such as bacteria and yeasts. It may also be used to treat chronic inflammatory lesions or processes where inappropriately available iron is acting as a catalyst for the generation of damaging reactive oxygen species.

### BRIEF DESCRIPTION OF THE DRAWINGS

FIG. 1 shows the plot obtained for ORC cloth (SURGICEL);

FIG. 2 shows the plot obtained for collagen/ORC [55/45] sponge;

FIG. 3 shows the plot obtained for collagen/alginate sponge (FIBRACOL); and

FIG. 4 shows the plot obtained for collagen sponge.

### DETAILED DESCRIPTION OF THE INVENTION

We have found, surprisingly, that oxidised cellulose exhibits a significant capacity to bind iron from an aqueous environment without the need to have attached to it a substance such as desferrioxamine known to have this property. Oxidised cellulose may be combined with one or more other biopolymers, for example collagen, in order to provide a composition that remains substantially insoluble in an aqueous environment. We were further surprised to find that the combination of oxidised cellulose with other biopolymers does not necessarily lead to the abrogation of its iron binding activity.

The term “oxidised cellulose” refers to any material produced by the oxidation of cellulose, for example with nitrogen dioxide/nitrogen tetraoxide (NO₂/N₂O₄). Such oxidation with NO₂/N₂O₄ converts primary alcohol groups at the C-6 position on the saccharide residues to carboxylic acid groups, forming glucuronic acid residues within the cellulose chain. As a secondary event, a dehydration reaction leads to lactone formation between the carboxylic acid group and a secondary hydroxyl group on the same saccharide residue. These lactones are alkali labile, and at pH 7 or higher their hydrolysis initiates the decomposition of the polymer via sugar ring cleavage. As a result, oxidised cellulose is biodegradable and bioabsorbable under physiological conditions. The preparation and properties of oxidised cellulose are described more fully by Stillwell R L and co-authors on pp. 291-306 in the Handbook of Biodegradable Polymers, a volume edited by Domb A J et al. and published by Harwood Academic Publishers of Amsterdam in 1997.

It is believed that the previously unknown iron binding activity of oxidised cellulose is a function of serendipitously appositioned carboxylate, hydroxyl and lactone functions. When oxidised cellulose is combined with other biopolymers, for example collagen, ion pairing between carboxylate groups on the oxidised cellulose and amino groups on the collagen may be expected to reduce or eliminate the iron binding activity. We have found that the combination of oxidised cellulose with collagen neither eliminates nor reduces the iron binding activity of oxidised cellulose.

It is an object of the present invention to provide a biologically acceptable solid composition showing a significant capacity to bind, chelate or sequester iron from an aqueous environment whilst itself being substantially insoluble in that environment.

It is a further object of the present invention to provide medical or clinical uses of such an iron-binding solid composition in the prevention or treatment of infection or of chronic inflammatory conditions arising from the iron-catalysed generation of damaging reactive oxygen species.

The present invention provides a porous solid, substantially insoluble in water, containing at least 25% by weight of an oxidised cellulose together with one or more other biopolymers or other suitable materials that, when combined with the oxidised cellulose, do not abrogate its iron binding capacity.

The present invention also provides a method of sequestering dissolved iron from an aqueous solution by bringing into contact with one another, the solution and said porous solid containing at least 25% by weight of an oxidised cellulose.

The present invention further provides the use of a porous solid, substantially insoluble in water, containing at least 25% by weight of an oxidised cellulose together with one or more other biopolymers or other suitable materials for the preparation of a device for the medical or clinical prevention or treatment of a condition caused in whole or in part by inappropriately available iron. Typically, the condition is an infection caused by one or more micro-organisms, for example bacteria or yeasts. Alternatively, the condition is a chronic inflammatory lesion or process where the inappropriately available iron is acting as a catalyst for the generation of damaging reactive oxygen species.

In a further aspect the present invention provides a method for prevention or treatment of a bacterial, yeast or similar such infection in a mammal comprising administering or applying a therapeutically effective amount of said porous solid containing at least 25% by weight of an oxidised cellulose.

The preferred oxidised cellulose for practical applications is oxidised regenerated cellulose (ORC) prepared by oxidation of a regenerated cellulose, such as rayon. It has been known for some time that ORC has haemostatic properties. ORC has been available as a haemostatic product called SURGICEL (Registered Trade Mark of Johnson & Johnson Medical, Inc.) since 1950. This product is produced by the oxidation of a knitted rayon material.

A modification of porosity, density and knit pattern led to the launch of a second ORC fabric product called INTERCEED (Registered Trade Mark of Johnson & Johnson Medical, Inc.), which was shown to reduce the extent of post-surgical adhesions in abdominal surgery.

WO98/00180 describes the use of ORC and complexes thereof for the treatment of chronic wounds, such as diabetic ulcers. The mechanism of action of the ORC in chronic wound treatment is thought to involve binding and inactivation of matrix metalloproteinase enzymes present in the wound fluid.

WO98/00446 describes the preparation of ORC oligosaccharides by partial hydrolysis of ORC in alkaline solution, followed by dialysis and purification. The ORC oligosaccharides are shown to have similar matrix metalloproteinase binding properties to ORC, and are also indicated for the treatment of chronic wounds.

In the use according to the present invention, the oxidised cellulose preferably comprises oxidised regenerated cellulose. The ORC may be in the form of fibres or woven or non-woven fabrics or freeze-dried or solvent-dried sponges. Preferably, at least 40% by weight of the solid material consists of oxidised regenerated cellulose.

In preferred embodiments of the present invention, the oxidised cellulose is combined with collagen to form structures of the kind described in WO98/00180 and WO98/00446, the entire contents of which are expressly incorporated herein by reference. For example, the oxidised cellulose may be in the form of milled ORC fibres that are dispersed in a freeze-dried collagen sponge. This provides for certain therapeutic and synergistic effects arising from the combination with collagen.

Preferably, the solid composition containing oxidised cellulose according to the present invention is substantially insoluble in water. That is to say, it has a solubility of less than 1 g/l in water at 25° C. Low solubility renders such compositions especially suitable for use with biological fluids from which iron is required to be removed.

Preferably, the affinity for iron of a solid composition containing oxidised cellulose according to the present invention is such that it will reduce the quantity of free or weakly complexed iron in aqueous solution by a factor of 10², and preferably by at least 10³.

### BRIEF DESCRIPTION OF THE DRAWINGS

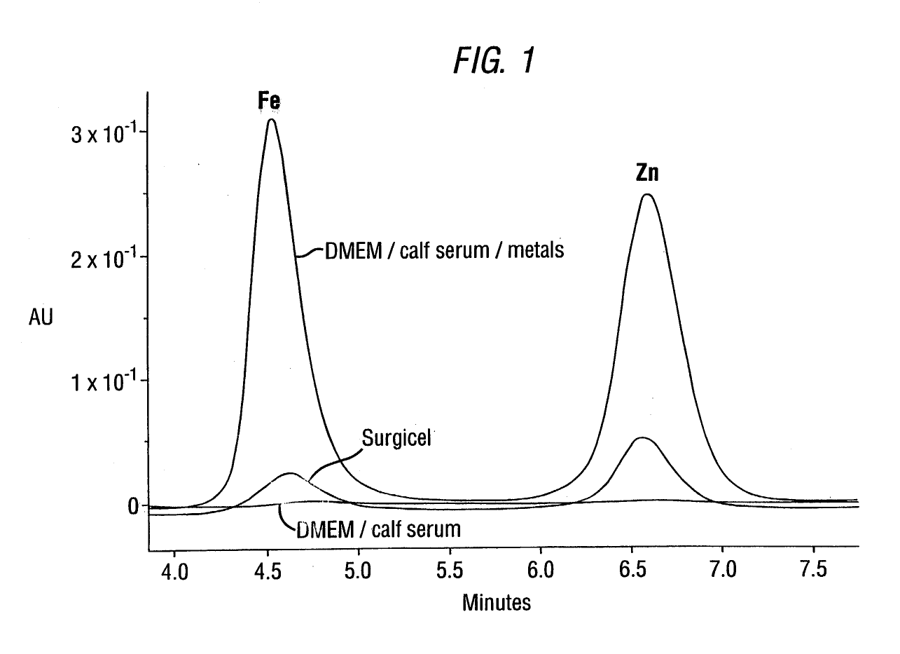

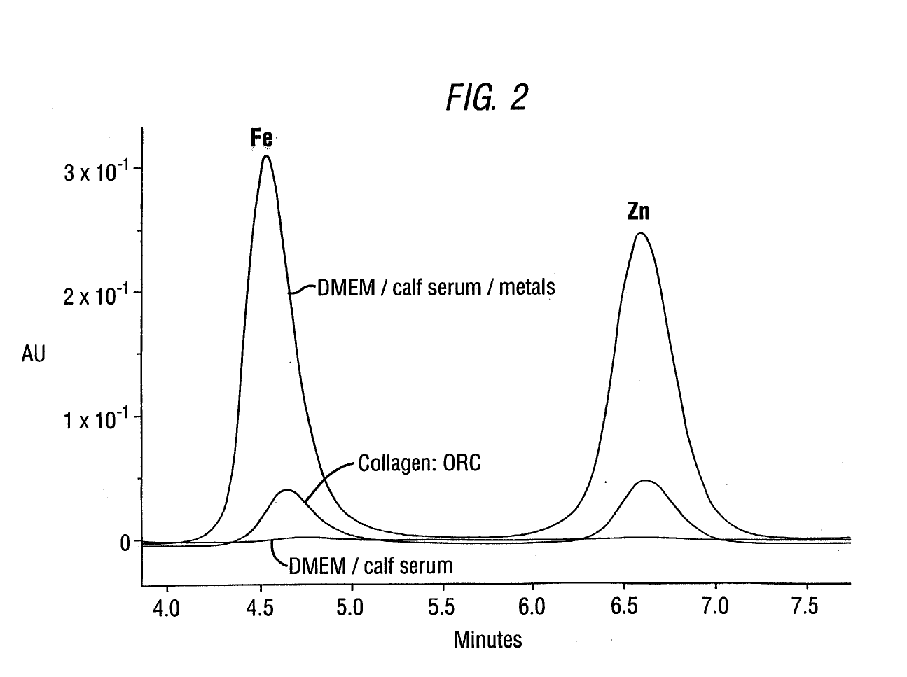

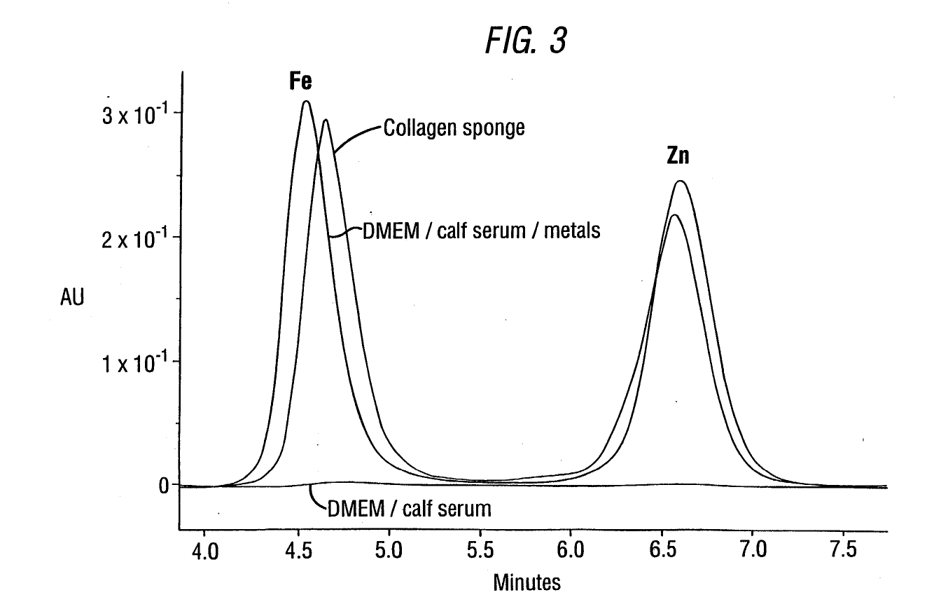

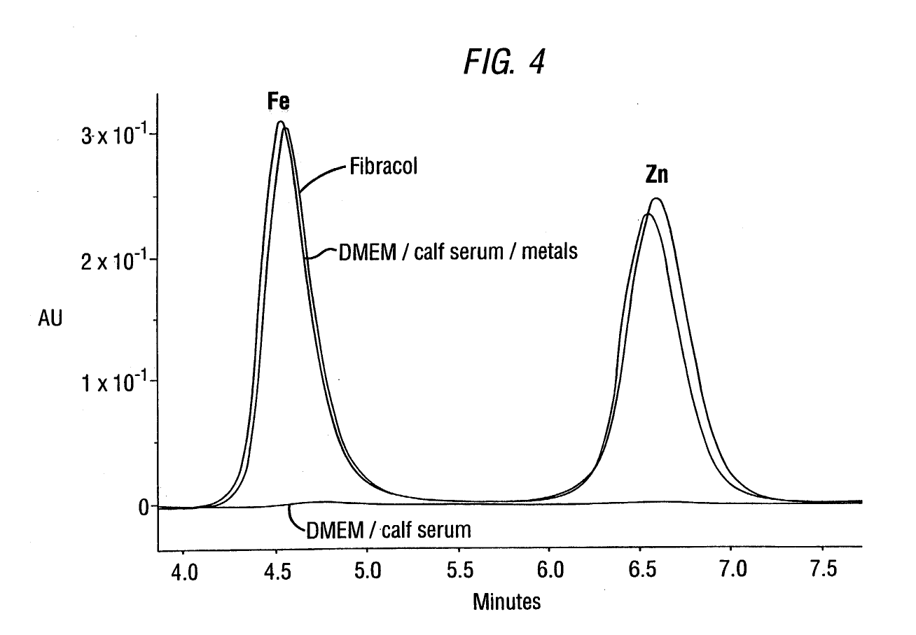

Particular embodiments of the present invention will now be described further, by way of example, with reference to the accompanying drawings. Thus, FIGS. 1-4 show Fe³⁺ peaks from ion exchange HPLC elution plots produced in accordance with Procedures 1 and 2 applied to Examples 1-4 respectively as described below. Each Figure shows two control plots, one being for Dulbecco's Modified Eagle's Medium (DMEM) containing 10% v/v calf serum, which is intended to simulate biological fluid, and the other being for DMEM containing 10% v/v calf serum to which has been added 50 ppm iron as ferric chloride, which is intended to simulate biological fluid containing decompartmentalised iron such as may occur in damaged tissue or chronic inflammatory lesions.

### DETAILED DESCRIPTION

### EXAMPLE 1

A sample of a commercially available knitted ORC cloth (registered trade mark SURGICEL of Johnson & Johnson Medical, Arlington) was provided.

### EXAMPLE 2

A collagen/ORC sponge was prepared in similar fashion to the method described in WO98/00180. Briefly, purified collagen was suspended in 0.05 M acetic acid and sufficient milled ORC powder (milled SURGICEL cloth) added to produce a weight ratio of 55/45 collagen/ORC and a total solids concentration of about 0.67% by weight. The mixture was then homogenised. The suspension was degassed in a vacuum oven for 10 minutes, and then poured into a tray and rapidly frozen at −40° C. The frozen suspension was then freeze-dried and dehydrothermally cross-linked using a programmable freeze-drier with a temperature ramping facility.

### EXAMPLE 3 (comparative)

A sample was obtained of a commercially available collagen/alginate sponge produced by freeze drying a slurry of collagen and alginate substantially as described in U.S. Pat. No. 4,614,794. The product is commercially available under the registered trade mark FIBRACOL from Johnson & Johnson Medical, Arlington.

### EXAMPLE 4 (comparative)

A freeze dried collagen sponge was prepared substantially as described in Example 2, but omitting the ORC from the slurry.

Procedure 1

The ability of the materials to bind iron in aqueous media according to the present invention was determined as follows:

The solid material was added with stirring to a solution containing iron (provided as Fe³⁺) in a serum-containing mammalian cell culture medium intended to simulate biological fluid. Following a suitable incubation period, ion exchange chromatography was used to determine the levels of uncomplexed Fe³⁺ remaining in solution.

The iron containing solution was prepared using FeCl₃.6H₂O to contain a final concentration of 50 ppm of iron dissolved in Dulbecco's Modified Eagle's Medium (DMEM) [Sigma Chemical Co., Poole, Dorset England; catalogue item D 6046] containing 10% v/v calf serum [Sigma Chemical Co., Poole, Dorset England; catalogue item C 6278]. This iron-containing solution (10 ml) was incubated with 100 mg of the solid material at 37° C. with gentle agitation on a shaker table for 16 hours. The samples were then centrifuged and a 0.9 ml sample of the supernatant solution was analysed for iron content according to Procedure 2.

Procedure 2

The centrifuged solution from Procedure 1 was treated with 0.1 ml of a 20% w/v solution of trichloroacetic acid (TCA) to give a final concentration of 2% w/v TCA. The tube was vortexed for 10 seconds and then centrifuged for 15 minutes or longer to remove solids. The supernatant solution (0.5 ml) was added to a clean dry HPLC vial to which was added 0.5 ml of 0.5 M nitric acid prepared in de-ionised water.

The samples were then analysed for free iron in a Dionex DX500 HPLC apparatus using the following method parameters:

| | |
|---|---|
| Column | IonPac CG5A Guard column (Dionex part no. |
| | 046104) |
| | IonPac CS5A Analytical column (Dionex part no. |
| | 046100) |
| Eluents | A - De-jonised water |
| | B - Metpac PDCA eluent concentrate (Dionex |
| | part no. 046088) |
| | C - Metpac PAR reagent diluent (Dionex |
| | part no. 046094) |
| Postcolumn Reagent | 4-(2-pyridylazo) resorcinol monosodium salt |
| | (PAR) 0.12 g/l |
| Detector Wavelength | 530 nm |
| Temperature | 25° C. |
| Flow rate | 1.2 ml/min (80% cluent A: 20% eluent B) in |
| | column; 0.6 ml/min eluent C postcolumn |
| Sample size | 100 μl |

Results

The results obtained for the samples tested (Examples 1-4) are shown in FIGS. 1-4. It is evident that the ORC cloth (Example 1) and the collagen/ORC [55/45] sponge (Example 2) remove iron effectively from solution whilst the collagen/alginate sponge (Example 3) and the collagen sponge (Example 4) both show little if any capacity to bind iron.

It will be known to those skilled in the art that the alginate present in Example 3 has a chemical structure not dissimilar to that of oxidised cellulose. Specifically, where alginate is a polysaccharide comprising mannuronic acid and guluronic acid residues, oxidised cellulose is a polysaccharide containing glucuronic acid residues. Mannuronic, guluronic and glucuronic acids all belong to the same class of glycuronic acids. Therefore, it might reasonably be expected that alginate/collagen sponge (Example 3) should show iron binding activity as seen with oxidised cellulose/collagen sponge (Example 2). It is evident from our results that this expectation was not fulfilled.

The above embodiments have been described by way of example only. Other embodiments falling within the scope of the accompanying claims will be apparent to the skilled reader.

1 A solid porous composition, substantially insoluble in water, comprising at least 25% by weight of an oxidised cellulose and exhibiting a significant capacity to remove iron from an aqueous environment.

2. A solid composition according to claim 1 wherein the oxidised cellulose consists essentially of oxidised regenerated cellulose.

3. A solid composition according to claim 1 wherein the solid material comprises a freeze-dried sponge of collagen and oxidised regenerated cellulose.

4. A solid composition according to claim 1 wherein the solid material consists essentially of a freeze-dried sponge of collagen and oxidised regenerated cellulose.

5. A method of sequestering dissolved iron from an aqueous solution by bringing the solution into contact with a solid porous composition that is substantially insoluble in water, and which contains at least 25% by weight of an oxidised cellulose.

6. A method according to claim 5 wherein the aqueous solution comprises a biological fluid.

7. A solid composition according to claim 5 wherein the oxidised cellulose consists essentially of oxidised regenerated cellulose.

8. A solid composition according to claim 5 wherein the solid material comprises a freeze-dried sponge of collagen and oxidised regenerated cellulose.

9. A solid composition according to claim 5 wherein the solid material consists essentially of a freeze-dried sponge of collagen and oxidised regenerated cellulose.

10. Use of a solid porous composition, substantially insoluble in water, comprising at least 25% by weight of an oxidised cellulose for the preparation of a device for the medical or clinical prevention or treatment of a condition caused in whole or in part by inappropriately available iron.

11. Use according to claim 10 wherein the medical or clinical condition comprises an infection caused by bacteria, yeasts or other iron-requiring micro-organisms.

12. Use according to claim 10 wherein the condition is a chronic inflammatory lesion or process where the inappropriately available iron is acting as a catalyst for the generation of damaging reactive oxygen species.

13. A solid composition according to claim 10 wherein the oxidised cellulose consists essentially of oxidised regenerated cellulose.

14. A solid composition according to claim 10 wherein the solid material comprises a freeze-dried sponge of collagen and oxidised regenerated cellulose.

15. A solid composition according to claim 10 wherein the solid material consists essentially of a freeze-dried sponge of collagen and oxidised regenerated cellulose.

16. A method for prevention micro-organism in a mammal comprising administering or applying a therapeutically effective amount of a porous solid that is substantially insoluble in water and contains at least 25% by weight of an oxidised cellulose.

17. A solid composition according to claim 16 wherein the oxidised cellulose consists essentially of oxidised regenerated cellulose.

18. A solid composition according to claim 16 wherein the solid material comprises a freeze-dried sponge of collagen and oxidised regenerated cellulose.

19. A solid composition according to claim 16 wherein the solid material consists essentially of a freeze-dried sponge of collagen and oxidised regenerated cellulose.
